(19) United States
(12) Plant Patent Application Publication
(10) Pub. No.: US 2018/0199481 P1
(43) Pub. Date: Jul. 12, 2018

(54) Vaccinium corymbosum L. plant named 'RYOKU NH-11'

(50) ;

(71) Applicant: NIPPON RYOKUSAN CO., LTD., Matsumoto-shi (JP)

(72) Inventor: Shigetaka Sakurai, Matsumoto-shi (JP)

(73) Assignee: NIPPON RYOKUSAN CO., LTD., Matsumoto-shi (JP)

(21) Appl. No.: 15/732,724

(22) Filed: Dec. 21, 2017

(30) Foreign Application Priority Data
Jan. 4, 2017 (JP) 31723

(51) Int. Cl. A01H 5/08

(52) U.S. Cl. PLT/157

(57) ABSTRACT

A new and distinct variety of Vaccinium corymbosum L. plant named ‘RYOKU NH-11’, characterized by having stronger plant vigor, a more upright plant growth habit, comparatively early fruit ripening time, comparatively large and uniform fruit size, and smaller and dry stem scar, as compared to other Vaccinium corymbosum L. varieties.

### CROSS-REFERENCE TO RELATED APPLICATIONS

This application claims priority to Japanese Plant Breeders' Rights Application No. 31723, filed Jan. 4, 2017, the contents of which are incorporated herein by reference in their entirety.

The Latin name of the genus and species of the novel variety disclosed herein is: Vaccinium corymbosum L.

The novel variety of the Vaccinium corymbosum L. disclosed herein has been given the variety denomination: ‘RYOKU NH-11’.

### BACKGROUND OF THE INVENTION

The present invention relates to a new and distinct hybrid variety of northern highbush blueberry (Vaccinium corymbosum L.) named ‘RYOKU NH-11’. This novel variety was found by open pollination of ‘Chandler’, a seed parent variety, in the tests conducted for the period from 2003 to 2007 in Matsumoto-City, Nagano-prefecture, Japan. As stated below, ‘RYOKU NH-11’ has apparently different characteristics from those of the varieties ‘Chandler’ and ‘Blueray’, both being widely planted and being important varieties in the Chubu district of Japan.

### SUMMARY OF THE INVENTION

Blueberry variety ‘RYOKU NH-11’ exhibits outstanding and distinguishing characteristics when grown under normal horticultural conditions in the area from Nagano-prefecture to the north of the Kanto in Japan, including:
(1) stronger plant vigor and more upright plant growth habit;
(2) comparatively early fruit ripening time (on average, around July 15 of each year, in Matsumoto-city, Nagano-prefecture, Japan);
(3) comparatively large and uniform fruit size; and
(4) smaller and dry stem scar.

### BRIEF DESCRIPTION OF THE DRAWINGS

The accompanying colored photographs (FIGS. 1 to 8) show typical bush, flower, fruit and leaf characteristics for the new Vaccinium corymbosum L. plant ‘RYOKU NH-11’. Colors shown are as accurate as can be reasonably reproduced by photographic means. In some cases, the color might differ slightly from the colors of ‘RYOKU NH-11’ recited in the description.

FIG. 1 shows a tree body of ‘RYOKU NH-11’ (photographed date: Jul. 10, 2015; photographed location: NIPPON RYOKUSAN CO., LTD; Matsumoto-City, Nagano-prefecture, Japan).

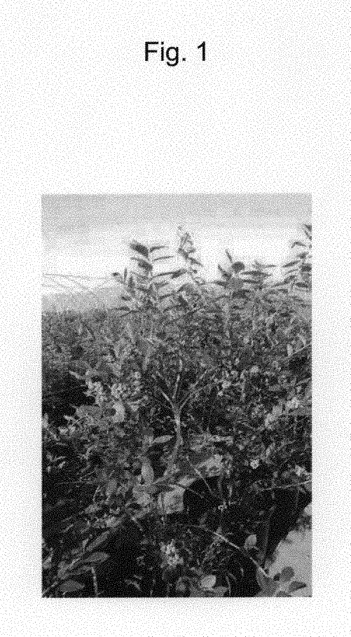

FIG. 2 shows a panoramic view of the cultivation area of ‘RYOKU NH-11’ (photographed date: Sep. 7, 2012; photographed location: same as FIG. 1).

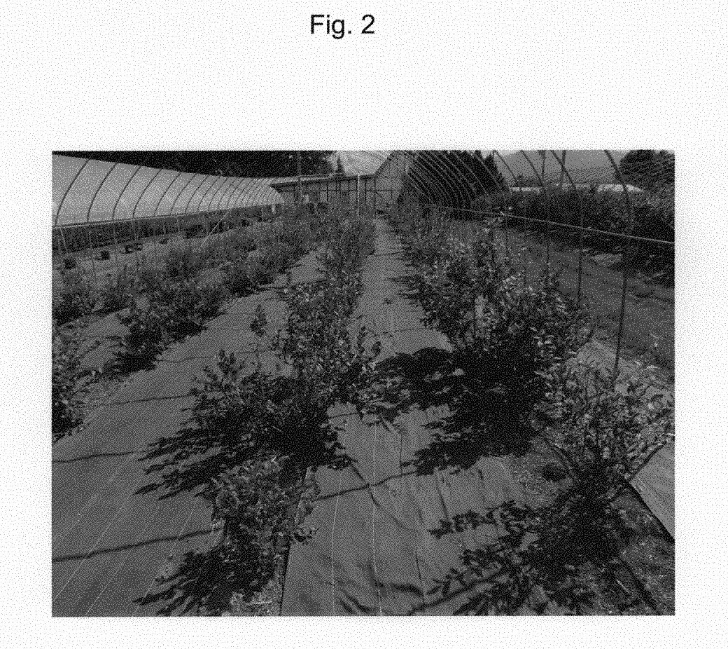

FIG. 3 shows whole flowers of ‘RYOKU NH-11’ (photographed date: May 6, 2014; photographed location: same as FIG. 1).

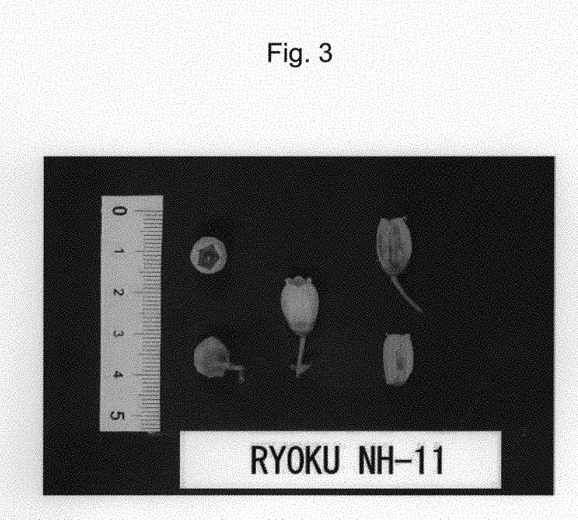

FIG. 4 shows broken-down parts of a flower of ‘RYOKU NH-11’ (photographed date: May 6, 2014; photographed location: same as FIG. 1).

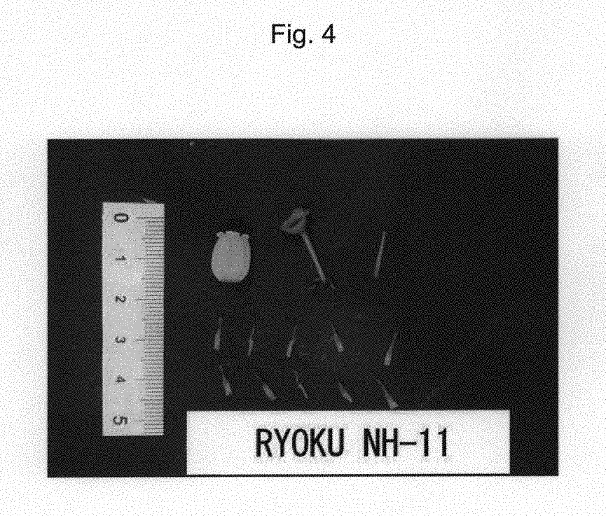

FIG. 5 shows fruits of ‘RYOKU NH-11’ (photographed date: Jul. 20, 2012; photographed location: same as FIG. 1).

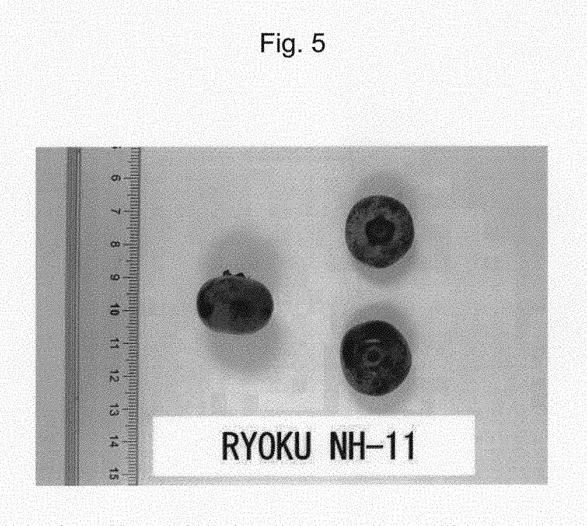

FIG. 6 shows a cross-section of the fruits of ‘RYOKU NH-11’ (photographed date: Jul. 20, 2012; photographed location: same as FIG. 1).

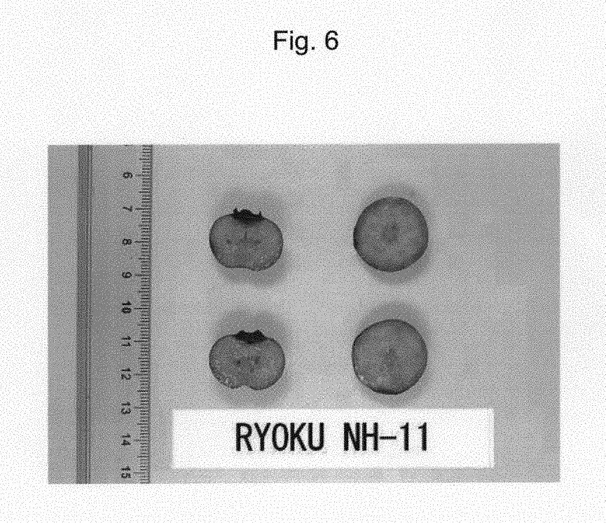

FIG. 7 shows an upper side of the leaves (10 sheets) of ‘RYOKU NH-11’ (photographed date: Aug. 3, 2012; photographed location: same as FIG. 1).

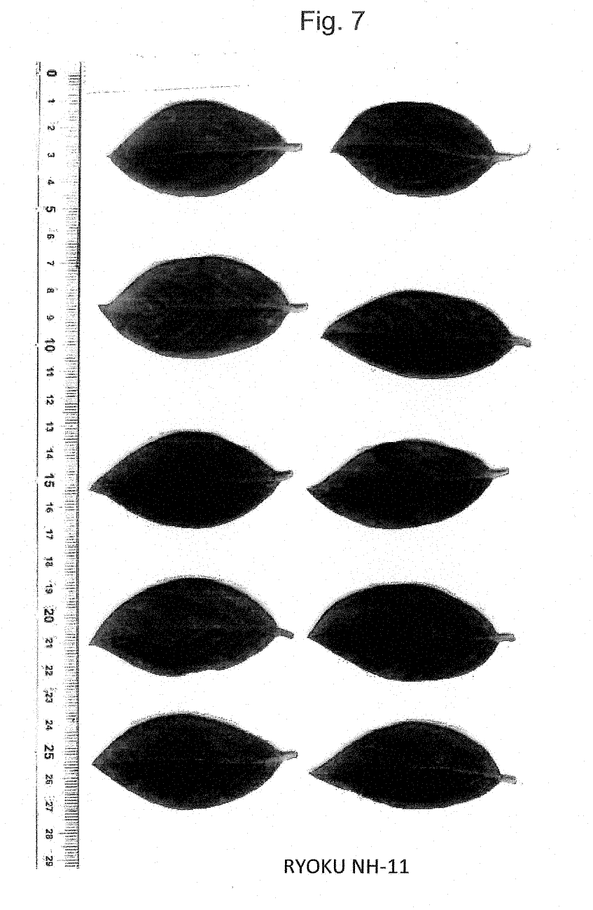

FIG. 8 shows a lower side of the leaves (10 sheets) of ‘RYOKU NH-11’ (photographed date: Aug. 3, 2012; photographed location: same as FIG. 1).

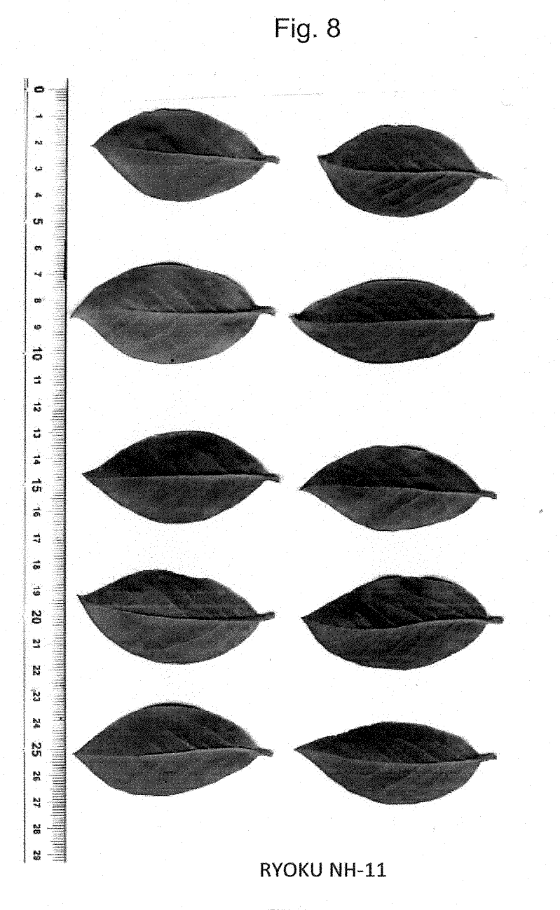

### DETAILED BOTANICAL DESCRIPTION

### A. Distinctive Characteristics of ‘RYOKU NY-11’

As described above, ‘RYOKU NH-11’ was obtained by the open pollination of ‘Chandler’, a seed parent (Note: the possible pollen parent might be ‘Blueray’). On Apr. 15, 2003, about 10,000 seeds of ‘Chandler’, which were cultivated in the field of NIPPON RYOKUSAN CO., LTD. (Matsumoto, Nagano, Japan), were seeded in plant seeding trays, and then transplanted to pots. The resulting seedlings (about 3,000) were planted in cultivation pots on May 1, 2004. Fructification of the planted seedlings were started from Jun. 20, 2007 (on Year 3), and about 200 plant individuals were selected based on the characteristics, including larger fruit size, better taste balance of sweetness and acidity, higher yield per plant, earlier ripening time, large and uniform fruits, etc. For the about 200 plant individuals selected, test plots (each including about 20 cuttings per plant individual) were formed, where these plants were asexually propagated by cutting means. During the period from Jul. 20, 2009 to Dec. 25, 2016 and for 3 generations, the plants were propagated and examined for their characteristics based on the growth, yield, and fruits quality in each test plot. For 10 test plots, the characteristics of the plants were observed for the period between the flowering time and the ripening time yearly for 5 years, and since neither variant nor off-type plant was observed for the period, the characterization of ‘RYOKU NH-11’ was finished on Dec. 25, 2016 and the breeding was completed.

‘RYOKU NH-11’ is a blueberry clone distinguishable from the important blueberry varieties ‘Chandler’ and ‘Blueray’, both of which are widely planted in the Chubu district of Japan, due to its characteristics including stronger plant vigor, more upright plant growth habit, earlier fruit ripening time, larger and more uniform fruit size, and smaller and dry stem scar. 27 plants of ‘RYOKU NH-11’ had been propagated by cutting means in Matsumoto, Nagano, Japan, and all the resulting plants were phenotypically indistinguishable from the original plant variety ‘RYOKU NH-11’. In addition, comparing to ‘Chandler’ and ‘Blueray’, the claimed plant ‘RYOKU NH-11’ has a more upright plant growth habit, about 1-week earlier fruit ripening time (on average, around July 15 of each year in Matsumoto, Nagano, Japan), more uniform fruit size and smaller stem scar when compared to its related variety ‘Chandler’, and has stronger plant vigor, larger fruit size, and smaller stem scar when compared to the ‘Blueray’ variety (see Table 1 below).

The following data defining the characteristics of ‘RYOKU NH-11’ were collected from the asexual propagation carried out in Matsumoto, Nagano, Japan. The plant history was taken on a plot of 10 five-year-old plants growing in Matsumoto, Nagano, Japan. ‘RYOKU NH-11’ has not been observed under all possible environmental conditions, and the measurements provided might therefore vary if grown in different environments. Where averages are given, the sample size was 10.

B. Phenotypic Description of Vaccinium corymbosum L. (‘RYOKU NH-11’)

Characteristics of ‘RYOKU NH-11’ are further specifically described as follows:
1. Plant:
   Plant vigor.—Strong, where the plant vigor is stronger than ‘Blueray’.
   Plant size.—Large.
   Growth habit.—Upright.
   Plant height.—1.7 m on average for 5-year old plant.
   Plant spread.—1.1 m on average for 5-year old plant.
   Tendency toward evergreenness.—Absent.
   Cold hardiness.—Survived in winter frost (below −10° C.) with minimum damage.
   Ease of propagation.—At least 70% rooting percentage in each of the dormant wood cutting and softwood stem cutting, comparable to the other varieties.
2. Trunk and branches:
   Suckering tendency.—Less suckering as in ‘Chandler’.
   Surface texture (of 6-month-old shoots).—Medium smoothness.
   Surface texture (of 3-year-old and older wood).—Medium smoothness.
   Color of new twigs observed in the field.—Yellow green.
   Internode length.—15.3 mm on average.
3. Leaves:
   Length including petiole.—69.4 mm on average.
   Width at widest point.—35.2 mm on average.
   Shape.—Elliptic.
   Leaf margin.—Entire.
   Color.—Upper surface: Medium Green.
   Pubescence.—Upper Surface of leaves: Absent. Lower Surface of leaves: Absent. Margins: Absent.
   Timing of vegetative bud burst.—Medium.
4. Flowers:
   Shape.—Campanulate.
   Flowering period.—Mean date of 50% opening of flowers in Matsumoto-City, Nagano-prefecture, Japan is May 1 (2 days earlier than ‘Chandler’).
   Corolla.—Diameter: 9.6 mm on average. Length (from pedicel attachment point to corolla tip excluding the pedicel): 12.3 mm on average. Color: light yellow white. Anthocyanin coloration in corolla tube — Absent or very weak.
5. Reproductive organs:
   Pollen.—Color: Yellow.
6. Fruit:
   Mean date of 50% harvest in Matsumoto-City, Nagano-prefecture.—July 15.
   Diameter of calyx aperture on mature berry.—7.2 mm on average.
   Size and shape of calyx lobe on mature berry.—Medium in size, outcurving, and having deep calyx basin.
   Detachment force for ripe berries (easy, medium, hard).—Easy.
   Fruit cluster density (sparse, medium, dense).—Medium.
   Fruiting type.—On one-year old shoots only.
7. Berry:
   Cluster (tight, medium, loose).—Medium.
   Weight (on well-pruned plants).—4.51 g on average.
   Height.—15.4 mm on average.
   Width.—22.0 mm on average.
   Shape.—Oblate.
   Surface color of mature berries ripe on the plant.—Blue purple.
   Intensity of fruit bloom.—Medium.
   Surface color of ripe berry after polishing.—Medium blue.
   Immature berry color, with bloom.—Light green.
   Immature berry color, without bloom.—Light yellow green.
   Flesh color.—Light yellow to light green.
   Peel color.—Medium blue.
   Pedicel scar.—Medium, 2.54 mm on average.
   Firmness.—Medium.
   Intensity of fruit sweetness.—Medium, Bx 10.8.
   Intensity of fruit acidity.—High, pH 2.80.
   Texture.—Crispy and juicy, medium seeds.
8. Use: ‘RYOKU NH-11’ produce northern highbush blueberries suitable for fruit-picking farms, fresh fruit markets and processed fruit markets, etc.
9. Resistance to disease, insects, and mites: ‘RYOKU NH-11’ grew vigorously and showed excellent bush survival in the field. It appears to be tolerant to stem blight (Boliyosphaeria spp.) and root rot (Phytophthora cinnamoni), with very few young plants dying soon after planting. The response of ‘RYOKU NH-11’ to the various fungal species that cause summer leaf spots is typical of other northern highbush varieties, and fungicide applications may be needed after harvest in order to reduce foliar diseases and to retain leaves until autumn and make maximum flower bud set. Similarly, susceptibility to typical blueberry insect and mite pathogens, such as spotted wing drosophila (Drosophila suzukii), blueberry gall midge (Dasineura oxycoccana) and blueberry bud mite (Acalitus vaccini), is similar to other northern highbush cultivars.

TABLE 1
(Comparison of characteristics among varieties)

| | | | | | | | | | The present | | |
|---|---|---|---|---|---|---|---|---|---|---|---|
| | | | | | | | | Standard | variety | | |
| Charact. | UPOV | | | | | | | Variety | RYOKU | Control Varieties | |
| No. | No. | Code | Characteristics | Definition | Method | Class | State | (Ex. Var.) | NH-11 | Blueray | Chandler |
| 1 | 1 | QN | Plant: vigor | Strength of | Observation | 3 | weak | Bluetta, | 7 | 6 | 7 |
| | (*) | (+) | | growth level | (a) VG | | | Meader | | | |
| | | | | of plant | | 5 | medium | Collins, | | | |
| | | | | | | | | Weymouth | | | |
| | | | | | | 7 | strong | Berkeley, | | | |
| | | | | | | | | Homebell, | | | |
| | | | | | | | | Woodard | | | |
| 2 | | QN | Plant: size | Size of plant | Observation | 3 | small | Avonblue, | 7 | 5 | 7 |
| | | | | crown | (a) VG | | | Bluetta, | | | |
| | | | | | | | | Flordablue | | | |
| | | | | | | 5 | medium | Bluecrop, | | | |
| | | | | | | | | Earliblue | | | |
| | | | | | | 7 | large | Dixi, | | | |
| | | | | | | | | Homebell, | | | |
| | | | | | | | | Tifblue | | | |
| 3 | 2 | QN | Plant: growth | Whole shape of | Observation | 1 | upright | Becyblue, | 1 | 1 | 2 |
| | (*) | G | habit | plant without | (a) VG | | | Bluechip, | | | |
| | | | | pruning during | | | | June, | | | |
| | | | | dormant period | | | | Spartan | | | |
| | | | | | | 2 | semi-upright | Bluecrop, | | | |
| | | | | | | | | Lateblue | | | |
| | | | | | | 3 | spreading | Northland, | | | |
| | | | | | | | | Weymouth | | | |
| 4 | 3 | PQ | One-year-old | Color of middle | Observation | 1 | green | | 5 | 5 | 4 |
| | | | shoot: color | part of shoot | (a) VG | 2 | greenish red | | | | |
| | | | | extended before | | 3 | greyish red | Briteblue, | | | |
| | | | | dormant period | | | | Homebell | | | |
| | | | | | | 4 | reddish | Berkeley, | | | |
| | | | | | | | yellow | Dixi | | | |
| | | | | | | 5 | reddish | Blueray, | | | |
| | | | | | | | brown | Darrow, | | | |
| | | | | | | | | Weymouth | | | |
| | | | | | | 6 | dark red | | | | |
| 5 | | QN | One-year-old | Length of | Measurement | 3 | short | | 6 | 5 | 7 |
| | | | shoot: length | middle part of | mm | 5 | medium | | (214 mm) | (173 mm) | (288 mm) |
| | | | | shoot extended | (a) | 7 | long | | | | |
| | | | | before dormant | VG | | | | | | |
| | | | | period | | | | | | | |
| 6 | 4 | QN | One-year-old | Length of | Observation | 3 | short | Avonblue, | 4 | 3 | 5 |
| | | | shoot: length | internode of | (a) VG | | | Weymouth | (15.3 mm) | (14.0 mm) | (16.7 mm) |
| | | | of internode | shoot extended | | 5 | medium | | | | |
| | | | (upper half) | before dormant | | 7 | long | Jersey | | | |
| | | | | period (upper | | | | | | | |
| | | | | half) | | | | | | | |
| 7 | 5 | QN | Leaf: length | Length of leaf | Measurement | 3 | short | | 5 | 5 | 5 |
| | (*) | | | sufficiently | mm | 5 | medium | | (69.4 mm) | (68.0 mm) | (67.0 mm) |
| | | | | expanded | (b) MS/VG | 7 | long | | | | |
| 8 | 6 | QN | Leaf: width | Maximum | Measurement | 3 | narrow | | 5 | 5 | 5 |
| | | | | width of | mm | 5 | medium | | (35.2 mm) | (31.0 mm) | (36.3 mm) |
| | | | | mature leaf | (b) MS/VG | 7 | broad | | | | |
| 9 | 7 | QN | Leaf: ratio | Ratio of leaf | Measurement | 3 | small | | 5 (1.97) | 5 (2.20) | 5 (1.85) |
| | | | length/width | length to | (b) MS/VG | 5 | medium | | | | |
| | | | | maximum | | 7 | large | | | | |
| | | | | width (leaf | | | | | | | |
| | | | | length/leaf | | | | | | | |
| | | | | width) | | | | | | | |
| 10 | 8 | PQ | Leaf: shape | Shape of | Observation | 1 | lanceolate | | 3 | 3 | 3 |
| | (*) | | | mature leaf | (b) VG | 2 | ovate | Northland | | | |
| | | | | | | 3 | elliptic | Berkeley, | | | |
| | | | | | | | | Collins, | | | |
| | | | | | | | | Coville | | | |
| | | | | | | 4 | oblong | | | | |
| 11 | | QN | Leaf: shape of | Shape of tip of | Observation | 3 | acute | Weymouth, | 5 | 5 | 5 |
| | | (+) | tip | mature leaf | (b) VG | | | Woodard | | | |
| | | | | | | 5 | medium | Earliblue, | | | |
| | | | | | | | | Tifblue | | | |
| | | | | | | 7 | obtuse | Berkeley, | | | |
| | | | | | | | | Climax, | | | |
| | | | | | | | | Southland | | | |
| 12 | 9 | QL | Leaf: color of | Color of surface | Observation | 1 | yellow | | 2 | 2 | 2 |
| | | | upper side | of mature leaf | (b) VG | 2 | green | Bluechip, | | | |
| | | | | | | | | Bluecrop, | | | |
| | | | | | | | | Blueray | | | |
| 13 | 10 | QN | Only varieties | Intensity of | Observation | 3 | light | | 5 | 5 | 5 |
| | (*) | | with green leaf | green color on | (b) VG | 5 | medium | | | | |
| | | | color: Leaf: | surface of | | 7 | dark | | | | |
| | | | intensity of | mature leaf | | | | | | | |
| | | | green color on | | | | | | | | |
| | | | upper side | | | | | | | | |
| 14 | 11 | QL | Leaf: margin | Type of margin | Observation | 1 | entire | | 1 | 1 | 1 |
| | (*) | | | of mature leaf | (b) VG | 2 | serrate | | | | |
| 15 | 12 | QN | Flower bud: | Intensity of | Observation | 3 | weak | | 5 | 7 | 5 |
| | | | anthocyanin | anthocyanin | (a) VG | 5 | medium | | | | |
| | | | coloration | coloration of | | 7 | strong | | | | |
| | | | | flower bud | | | | | | | |
| | | | | occurring to | | | | | | | |
| | | | | one year old | | | | | | | |
| | | | | shoot | | | | | | | |
| 16 | 13 | QN | Inflorescence: | Length of | Measurement | 3 | short | | 6 | 5 | 6 |
| | | | length | inflorescence at | mm (c) | 5 | medium | | (35.6 mm) | (28.7 mm) | (35.1 mm) |
| | | | (excluding | flowering time | MS/VG | 7 | long | | | | |
| | | | peduncle) | (excluding | | | | | | | |
| | | | | peduncle) | | | | | | | |
| 17 | 14 | PQ | Flower: shape | Shape of corolla | Observation | 1 | urceolate | Bluecrop, | 2 | 1-2 | 2-3 |
| | | | of corolla | at full bloom | (c) VG | | | Jersey | | | |
| | | | | | | 2 | campanulate | Northblue, | | | |
| | | | | | | | | Northsky | | | |
| | | | | | | 3 | cylindrical | | | | |
| 18 | | PQ | Flower: color | Color of corolla | Observation | 1 | white | Aliceblue, | 2 | 3 | 1 |
| | | | of corolla | at full bloom | (c) VG | | | Bluetta, | | | |
| | | | | | | | | Briteblue | | | |
| | | | | | | 2 | creamy | Avonblue, | | | |
| | | | | | | | white | Berkeley, | | | |
| | | | | | | | | Bluecrop | | | |
| | | | | | | 3 | greenish | Blueray, | | | |
| | | | | | | | white | Collins, | | | |
| | | | | | | | | Coville | | | |
| | | | | | | 4 | light pink | Bluebell, | | | |
| | | | | | | | | Delite, | | | |
| | | | | | | | | Dixi, | | | |
| | | | | | | | | Tifblue | | | |
| 19 | 15 | QN | Flower: size | Size of corolla | Observation | 3 | small | | 7 | 5 | 7 |
| | (*) | | of corolla tube | tube at full | (c) VG | 5 | medium | | | | |
| | | | | bloom | | 7 | large | | | | |
| 20 | 16 | QN | Flower: | Intensity of | Observation | 1 | absent or | | 1 | 3 | 1 |
| | (*) | | anthocyanin | anthocyanin | (c) VG | | very weak | | | | |
| | | | coloration of | coloration on | | 3 | weak | | | | |
| | | | corolla tube | surface of | | 5 | medium | | | | |
| | | | | corolla tube | | 7 | strong | | | | |
| 21 | 17 | QL | Flower: ridges | Presence or | Observation | 1 | absent | Herbert | 9 | 9 | 9 |
| | | | on corolla tube | absence of | (c) VG | 9 | present | Aliceblue | | | |
| | | | | ridges on | | | | Berkeley, | | | |
| | | | | corolla tube | | | | Dixi | | | |
| 22 | 18 | QN | Fruit cluster: | Density of fruit | Observation | 3 | sparse | Homebell, | 5 | 7 | 7 |
| | | | density | per fruit cluster | (d) VG | | | Jersey, | | | |
| | | | | | | | | Woodard | | | |
| | | | | | | 5 | medium | Bluechip, | | | |
| | | | | | | | | Bluecrop, | | | |
| | | | | | | | | Bluetta | | | |
| | | | | | | 7 | dense | Darrow, | | | |
| | | | | | | | | Herbert, | | | |
| | | | | | | | | Patriot | | | |
| 23 | 19 | QN | Unripe fruit: | Intensity of | Observation | 3 | light | | 3 | 7 | 3 |
| | (*) | | intensity of | green color of | VG | 5 | medium | | | | |
| | | | green color | fruit before | | 7 | dark | | | | |
| | | | | ripening | | | | | | | |
| 24 | 20 | QN | Fruit: size | Size of fruit at | Observation | 3 | small | Home ball | 7 | 5 | 7 |
| | (*) | | | ripening | (d) VG | | | June, | (4.51 g) | (2.89 g) | (5.72 g) |
| | | | | | | | | Northblue | | | |
| | | | | | | 5 | medium | Collins, | | | |
| | | | | | | | | Earliblue | | | |
| | | | | | | 7 | large | Berkeley, | | | |
| | | | | | | | | Bluecrop, | | | |
| | | | | | | | | Spartan | | | |
| 25 | 21 | PQ | Fruit: shape in | Shape in | Observation | 1 | elliptic | | 3 | 3 | 3 |
| | (*) | (+) | longitudinal | longitudinal | (d) VG | 2 | round | Berkeley, | | | |
| | | | section | section of fruit | | | | Jersey, | | | |
| | | | | at ripening | | | | Sharpblue | | | |
| | | | | | | 3 | oblate | Earliblue, | | | |
| | | | | | | | | Harison, | | | |
| | | | | | | | | Woodard | | | |
| 26 | | QN | Fruit: size of | Size of stem | Observation | 3 | small | | 5 | 7 | 7 |
| | | (+) | scar | scar of mature | (d) VG | 5 | medium | | (2.54 mm) | (3.80 mm) | (3.98 mm) |
| | | | | fruit | | 7 | large | | | | |
| 27 | | PQ | Fruit: shape of | Shape of calyx | Observation | 1 | star | | 2 | 2 | 2 |
| | | (+) | calyx cavity | cavity of mature | (d) VG | 2 | circular | | | | |
| | | | | fruit | | | | | | | |
| 28 | 22 | QN | Fruit: attitude | Attitude of | Observation | 1 | erect | | 3 | 3 | 3 |
| | | | of sepals | sepals relative | (d) VG | 2 | erect to | | | | |
| | | | | to mature fruit | | | semi-erect | | | | |
| | | | | | | 3 | semi-erect | | | | |
| | | | | | | 4 | level | | | | |
| 29 | 23 | QN | Fruit: type of | Direction of | Observation | 1 | incurving | | 3 | 1 | 3 |
| | | | sepals | curving of | (d) VG | 2 | straight | | | | |
| | | | | sepals | | 3 | reflexed | | | | |
| 30 | 24 | QN | Fruit: diameter | Diameter of | Observation | 3 | small | Avonblue, | 7 | 5 | 7 |
| | | | of calyx basin | calyx basin of | (d) VG | | | Bluechip, | (7.18 mm) | (4.40 mm) | (6.46 mm) |
| | | | | mature fruit | | | | Sharpblue | | | |
| | | | | | | 5 | medium | Blueray, | | | |
| | | | | | | | | Woodard | | | |
| | | | | | | 7 | large | Coville, | | | |
| | | | | | | | | Darrow, | | | |
| | | | | | | | | Homebell | | | |
| 31 | 25 | QN | Fruit: depth of | Depth of calyx | Observation | 3 | shallow | Bluecrop, | 7 | 7 | 7 |
| | | | calyx basin | basin of mature | (d) VG | | | Rancocas, | (2.40 mm) | (2.60 mm) | (2.32 mm) |
| | | | | fruit | | | | Tifblue | | | |
| | | | | | | 5 | medium | Earliblue, | | | |
| | | | | | | | | Jersey | | | |
| | | | | | | 7 | deep | Blueray, | | | |
| | | | | | | | | Collins, | | | |
| | | | | | | | | Dixi | | | |
| 32 | 26 | QN | Fruit: intensity | Intensity of | Observation | 1 | very weak | | 5 | 5 | 5 |
| | (*) | | of bloom | bloom on surface | (d) VG | 3 | weak | Dixi, | | | |
| | | | | of mature fruit | | | | Herbert, | | | |
| | | | | | | | | Sharpblue | | | |
| | | | | | | 5 | medium | Collins, | | | |
| | | | | | | | | Coville | | | |
| | | | | | | 7 | strong | Avonblue | | | |
| | | | | | | | | Bluecrop, | | | |
| | | | | | | | | Tifblue | | | |
| 33 | 27 | PQ | Fruit: color of | Color of skin | Observation | 1 | light blue | Berkeley, | 2 | 2 | 2 |
| | (*) | G | skin (after | of mature | (d) VG | | | Bluechip, | | | |
| | | | removal of | fruit after re- | | | | Tifblue | | | |
| | | | bloom) | moval of | | 2 | medium | Blueray, | | | |
| | | | | bloom | | | blue | Jerse, | | | |
| | | | | | | | | June | | | |
| | | | | | | 3 | dark blue | Dixi, | | | |
| | | | | | | | | Homebell | | | |
| | | | | | | 4 | blue red | | | | |
| 34 | 28 | QN | Fruit: firmness | Firmness of | Observation | 3 | soft | Herbert, | 5 | 6 | 5 |
| | | (+) | | mature fruit | (d) VG/VS | | | Homebell, | | | |
| | | | | | | | | Spartan | | | |
| | | | | | | 5 | medium | Collins, | | | |
| | | | | | | | | Dixi | | | |
| | | | | | | 7 | firm | Coville, | | | |
| | | | | | | | | Southland | | | |
| | | | | | | 9 | very firm | | | | |
| 35 | | PQ | Fruit: color of | Color of flesh of | Observation | 1 | white | Berkeley, | 2-3 | 3 | 3 |
| | | | flesh | mature fruit | (d) VG | | | Bluecrop, | | | |
| | | | | | | | | Blueray | | | |
| | | | | | | 2 | cream | Earliblue | | | |
| | | | | | | 3 | light green | Bluechip, | | | |
| | | | | | | | | Lateblue, | | | |
| | | | | | | | | Sharpblue | | | |
| | | | | | | 4 | light purple | Aliceblue, | | | |
| | | | | | | | | Delite, | | | |
| | | | | | | | | Homebell | | | |
| 36 | 29 | QN | Fruit: | Sweetness of | Observation | 3 | low | Avonblue, | 5 | 7 | 6 |
| | (*) | (+) | sweetness | mature fruit | (d) VG | | | Bluechip | (Bx 10.8) | (Bx 12.6) | (Bx 13.0) |
| | | | | | | 5 | medium | Berkeley, | | | |
| | | | | | | | | Bluetta, | | | |
| | | | | | | | | Spartan | | | |
| | | | | | | 7 | high | Aliceblue, | | | |
| | | | | | | | | Bluecrop, | | | |
| | | | | | | | | Blueray | | | |
| 37 | 30 | QN | Fruit: acidity | Acidity of | Observation | 3 | low | Earliblue, | 7 | 5 | 6 |
| | (*) | (+) | | mature fruit | (d) VG | | | Homebell | (pH 2.80) | (pH 3.12) | (pH 2.96) |
| | | | | | | 5 | medium | Blueray, | | | |
| | | | | | | | | Herbert | | | |
| | | | | | | 7 | high | Collins, | | | |
| | | | | | | | | Elliott, | | | |
| | | | | | | | | Lateblue | | | |
| 38 | 31 | QL | Plant: fruiting | Shoots to which | Observation | 1 | on one- | | 1 | 1 | 1 |
| | (*) | G | type | fruits adnate | (c) VG | | year-old | | | | |
| | | | | | | | shoots only | | | | |
| | | | | | | 2 | on one- | | | | |
| | | | | | | | year-old | | | | |
| | | | | | | | and current | | | | |
| | | | | | | | season's | | | | |
| | | | | | | | shoots | | | | |
| 39 | | QN | Fruit: | Tendency of | Observation | 3 | less | Earliblue, | 3 | 3 | 3 |
| | | | tendency of | cracking during | (d) VG | | | Herbert, | | | |
| | | | cracking | harvest season | | | | Spartan | | | |
| | | | | | | 5 | medium | Avonblue, | | | |
| | | | | | | | | Berkeley, | | | |
| | | | | | | | | Bluechip | | | |
| | | | | | | 7 | much | Briteblue, | | | |
| | | | | | | | | Climax, | | | |
| | | | | | | | | Darrow | | | |
| 40 | 32 | QN | Time of | Time of | Measurement | 3 | early | Avonblue, | 5 | 5 | 5 |
| | (*) | (+) | vegatative bud | beginning to | MG | | | Beckyblue, | Apr. 8 | Apr. 6 | Apr. 8 |
| | | | burst | burst first | | | | Sharpblue | (2016) | (2016) | (2016) |
| | | | | vegatative bud | | 5 | medium | Darrow, | | | |
| | | | | of each in- | | | | Weymouth | | | |
| | | | | dividual plant | | 7 | late | Elliott, | | | |
| | | | | | | | | Lateblue | | | |
| 41 | 33 | QN | Time of be- | Time of 10% | Measurement | 1 | very early | | 6 | 5 | 6 |
| | (*) | (+) | ginning of | flowering | MG | 3 | early | | Apr. 26 | Apr. 23 | Apr. 27 |
| | | G | flowering on | occurring to one | | 5 | medium | Bluecrop, | (2016) | (2016) | (2016) |
| | | | one-year-old | year old shoot | | | | Collins, | | | |
| | | | shoot | | | | | Woodard | | | |
| | | | | | | 7 | late | Dixi, | | | |
| | | | | | | | | Herbert, | | | |
| | | | | | | | | Lateblue | | | |
| | | | | | | 9 | very late | | | | |
| 42 | 34 | QN | Varieties | Time of 10% | Measurement | 3 | early | | — | — | — |
| | (*) | (+) | which fruit on | flowering | MG | 5 | medium | | | | |
| | | G | one-year-old | occurring to | | 7 | late | | | | |
| | | | shoot and | current year's | | | | | | | |
| | | | current | shoot | | | | | | | |
| | | | season's shoot: | | | | | | | | |
| | | | Time of | | | | | | | | |
| | | | beginning of | | | | | | | | |
| | | | flowering on | | | | | | | | |
| | | | current year's | | | | | | | | |
| | | | shoot | | | | | | | | |
| 43 | 35 | QN | Time of | Time of 10% | Measurement | 1 | very early | | 6 | 6 | 6 |
| | | (+) | beginning of | fruit ripening on | MG | 3 | early | Avonblue, | Middle | Middle | Late |
| | | G | fruit ripening | one year old | | | | Earliblue, | July to | July to | July to |
| | | | on one-year- | shoot | | | | Weymouth | late July | late July | early |
| | | | old shoot | | | 5 | medium | Herbert, | | | August |
| | | | | | | | | Jersey | | | |
| | | | | | | 7 | late | Briteblue, | | | |
| | | | | | | | | Elliott, | | | |
| | | | | | | | | Tifblue | | | |
| | | | | | | 9 | very late | | | | |
| 44 | | QN | Period of | Length of | Measurement | 3 | short | Darrow, | 5 | 5 | 5 |
| | | | harvest | harvest period | MG | | | Northblue | | | |
| | | | | of fruit | | 5 | medium | Berkeley | | | |
| | | | | | | | | Bluecrop | | | |
| | | | | | | 7 | long | Briteblue, | | | |
| | | | | | | | | Tifblue, | | | |
| | | | | | | | | Woodard | | | |
| 45 | 36 | QN | Varieties | Time of 10% | Measurement | 3 | early | | — | — | — |
| | (*) | (+) | which fruit on | fruit ripening on | MG | 5 | medium | | | | |
| | | G | one-year-old | current year's | | 7 | late | | | | |
| | | | shoot and | shoot | | | | | | | |
| | | | current | | | | | | | | |
| | | | season's shoot: | | | | | | | | |
| | | | Time of | | | | | | | | |
| | | | beginning of | | | | | | | | |
| | | | ripening on | | | | | | | | |
| | | | current year's | | | | | | | | |
| | | | shoot | | | | | | | | |

What is claimed is:

1. A new and distinct variety of Vaccinium corymbosum L. plant named ‘RYOKU NH-11’, as described and illustrated herein.
